(19) United States
(12) Patent Application Publication
(10) Pub. No.: US 2002/0068907 A1
(43) Pub. Date: Jun. 6, 2002

(54) Safety syringe with retraction trunk

(76) Inventor: Edward D. Dysarz, Rockport, TX (US)

Correspondence Address: STREETS & STEELE, 13831 Northwest Freeway, Suite 355, Houston, TX, 77040, US

(21) Appl. No.: 09/993,372

(22) Filed: Nov. 19, 2001

Related U.S. Application Data
(63) Continuation-in-part of application No. 09/654,668, filed on Sep. 5, 2000.

(51) Int. Cl. A61M 5/00

(52) U.S. Cl. 604/191

(57) ABSTRACT

Safety syringe with a syringe barrel and an exterior retraction trunk. The trunk is formed outside of the barrel, forming an exterior chamber fixed to the barrel. A needle cannula carriage is held within the distal end of the retraction trunk. A needle cannula module is fixed to the distal end of the needle cannula carriage, wherein a cannula provides fluid communication from the needle cannula module, through the needle cannula carriage, through the retraction trunk and into the syringe barrel. A plunger is provided in the inner surfaces of the syringe barrel and the outer surface of the retraction trunk. When desired, a button may be pushed causing the latching means to release the needle cannula carriage, allowing the biasing means to thrust said carriage with the needle cannula into the exterior retraction trunk, thereby preventing the needle cannula from accidentally pricking others or being reused.

This application is a continuation-in-part of U.S. patent application Ser. No. 09/654,668

### BACKGROUND OF THE INVENTION

1. Field of the Invention

The present invention relates to safety syringes having retractable needle cannulas.

2. Background of the Related Art

There are safety syringe devices of various designs that will allow the needle to retract into either the barrel or plunger of the syringe. Some of these devices are described in U.S. Pat. No 4,973,316 (Dysarz), U.S. Pat. No. 4,978,343 (Dysarz), U.S. Pat. No. 5,180,369 (Dysarz), U.S. Pat. No. 5,267,961 (Shaw), U.S. Pat. No. 5,019,044 (Tsao), U.S. Pat. No. 5,084,018 (Tsao), U.S. Pat. No. 5,385,551 (Shaw), U.S. Pat. No. 5,389,076 (Shaw), U.S. Pat. No. 5,201,710 (Caselli), U.S. Pat. No. 6,010,486 (Carter et al), U.S. Pat. No. 5,120,310 (Shaw), U.S. Pat. No. 5,188,613 (Shaw), and U.S. Pat. No. 6,033,385 (Liu). Although these devices reduce accidental needle sticks, an operator of the devices may cause the needle to prematurely retract during the process of filling the syringe (aspiration) or while medication is being injected into a body.

For example, during aspiration the plunger is pushed in as far as it will go into the syringe (just prior to pulling the plunger back to draw the medication or other fluid from a container or body) such that the plunger may trigger the retraction mechanism and cause the needle to prematurely retract, thereby resulting in the loss of the needle cannula, an expensive syringe, more expensive medication, and even more expensive labor (doctor, nurse, etc). Usually the operator of the device does not know at what precise point to stop pressing the plunger into the syringe prior to or during aspiration.

Other types of safety syringes are devices with a sleeve or a sheath that will cover the needle after it has been used. To aspirate or fill this type of syringe, one must sight the medication through two layers of plastic or glass; if a clear fluid is being drawn into the syringe it may not be possible to see the necessary dosage.

Additionally, most of the devices mentioned above are not modular, wherein a needle cannula of any size may be used with a syringe of any size. Rather, the needle cannula is made as part of the syringe, and the two are not interchangeable with other needle cannulas or syringes of different sizes or capacities. Safety syringes that are modular include the devices disclosed in U.S. Pat. No. 5,891,093 (Dysarz), U.S. Pat. No. 6,016,595 (Dysarz), U.S. Pat. No. 5,935,113 (Dysarz), and U.S. Pat. No. 6,010,486 (Carter et al).

Therefore, there remains a need for a safety syringe with a positive latching device that will remain latched during aspiration and that can only be released with a positive release means when the user desires to release the needle-latching device. Furthermore, there remains a need for a safety syringe that is modular, wherein various needle cannulas are interchangeable with various syringes. It would be desirable if the modular safety syringe were compatible with existing inventories of needles.

### SUMMARY OF THE INVENTION

The present invention provides a retractable safety syringe module, comprising a syringe barrel forming a fluid chamber, and an exterior retraction trunk. The exterior retraction trunk forms a chamber fixed to the syringe barrel, wherein the retraction trunk has a port in fluid communication with the syringe barrel.

The syringe barrel comprises a chamber and a plunger extending through a proximal end of the chamber, the plunger having a sliding gasket formed along its distal end for sealing against the interior walls of the chamber. A biased needle cannula carriage is releasably secured against the sealing collar, the biased needle cannula carriage having a connector for selectively receiving a needle cannula and a passage providing fluid communication between the sealing collar port and the connector. A retaining member releasably secures the biased needle cannula carriage against the sealing collar.

The needle cannula carriage is biased in the proximal direction into the retraction trunk and has dimensions allowing it to retract into the retraction trunk without restriction. A needle cannula is selectively and/or disconnectably connectable to the carriage, preferably by a connector selected from screw threads, Luer-Loks® (Becton Dickinson, Franklin Lakes, N.J.), snap-on fittings or slip-on fittings. An exterior surface of the carriage mates with the interior surface of the sealing collar, wherein the mating surfaces are preferably selected from shoulders, steps and cones. A retaining member, such as a carriage stop arm, is provided to secure the carriage against the sealing collar.

One aspect of the invention provides a kit of components for assembling safety syringes, the kit comprising two or more safety syringe modules, optionally having different diameters, and two or more needle cannula modules, optionally having different sizes. Each safety syringe module and each needle cannula module are provided in accordance with any embodiment described herein. The connectors in any of the two or more safety syringe modules are sealably securable to any of the connectors in the two or more needle cannula modules to provide fluid communication between the needle cannula and the passage in the carriage base. Specifically, the connectors are preferably selected from threads, Luer-Loks® (Becton Dickinson, Franklin Lakes, N.J.), snap-on fittings or slip-on fittings.

### BRIEF DESCRIPTION OF THE DRAWINGS

The features of the present invention can best be understood by reference to the following descriptions taken in conjunction with the accompanying drawings wherein like numerals indicate like parts.

FIG. 1 is a section elevation of the syringe of the first preferred embodiment.

FIG. 2 an enlarged section elevation view of the needle cannula carriage as taken from FIG. 1.

FIG. 3 is a section plan view of the needle cannula carriage as taken through FIG. 1.

FIG. 4 is a section plan view of the syringe, the needle cannula carriage and the retraction trunk as taken through FIG. 1.

FIG. 5 is a section elevation of the needle cannula carriage, and the needle cannula released and thrust into the retraction trunk.

FIG. 6 is a section elevation of the second preferred embodiment.

FIG. 7 is a section plan view of the needle cannula module connection with at least two syringe trunks fixed to the retraction trunk as taken through FIG. 6.

FIG. 8 is a section elevation of the device of the second preferred embodiment showing the needle carriage and needle cannula released.

FIG. 9 is a section elevation as taken through FIG. 8.

### DETAILED DESCRIPTION OF THE PREFERRED EMBODIMENTS

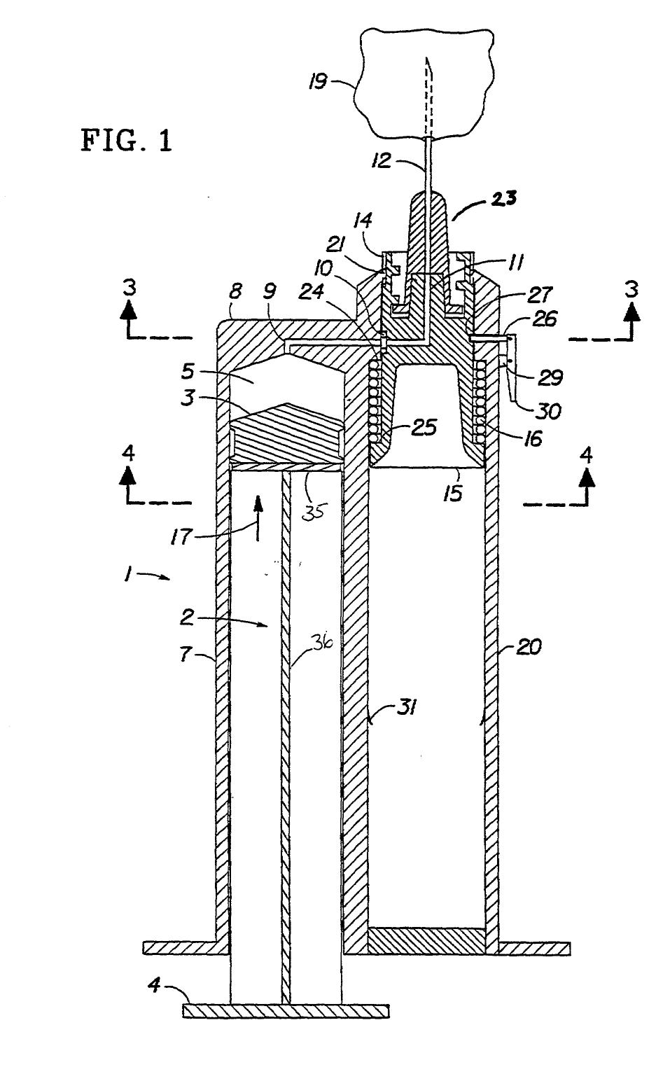

Referring to FIG. 1, there is shown a section elevation of a syringe 1 of the first preferred embodiment. The syringe is formed into an syringe barrel 7 with an inside surface and an outside surface, a distal end and a proximal end wherein the chamber end 8 forms a closure at the distal end of the syringe barrel. An elongated hollow passage or cannula 9 is shown formed in the chamber end and wherein the cannula 9 extends from the medication chamber 5 to the needle carriage 15.

A plunger 2 is shown inside the syringe barrel 7. The plunger has a plunger gasket 3 at the distal end of the plunger and a thumb flat 4 at the proximal end of the plunger. The plunger gasket 3 forms a fluid tight and gas tight seal around the inside surface of the syringe barrel 7. To actuate the plunger the thumb flat is depressed, thereby thrusting the plunger gasket in the distal direction 17 thereby creating pressure in the medication chamber 5, and further forcing the medication into syringe barrel cannula 9, past the carriage seal 10, into the carriage cannula 11, into the needle cannula 12 and into the body 19.

The carriage cannula 11 is shown formed in the needle carriage 15 that is shown in the retraction trunk 20. The retraction trunk 20 is suitably fixed adjacent to the syringe barrel 7. The retraction trunk preferably has at least one spline 21 formed at the distal end of the retraction trunk 20 wherein at least one spline meshes with at least one groove formed in the distal end of the needle carriage 14 to prevent the needle carriage 15 from rotating while the needle cannula base 23 is being threaded onto the needle carriage.

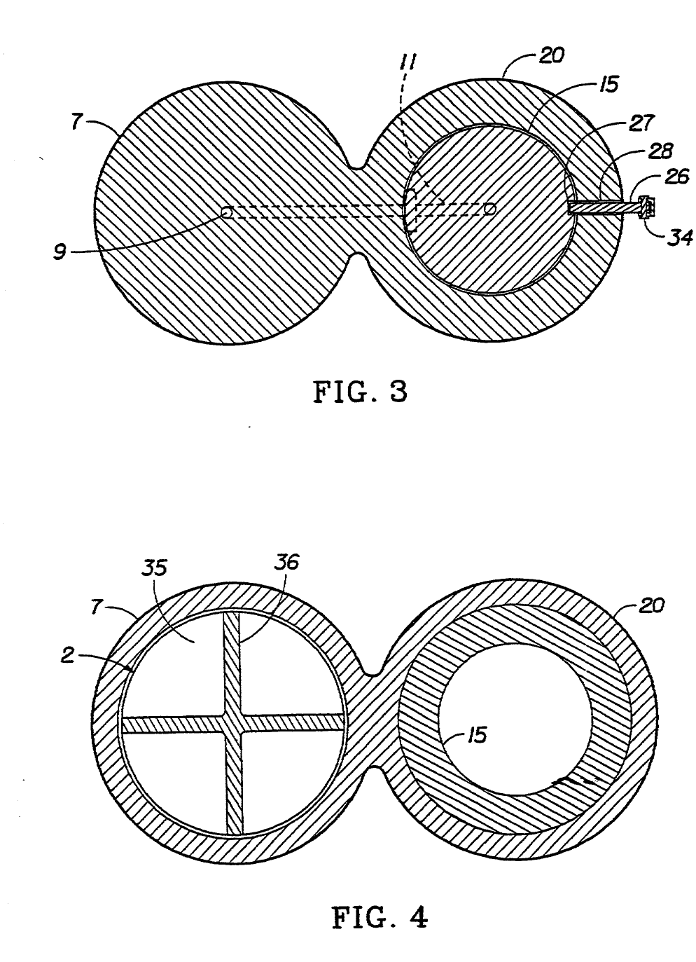

A spring ridge or shoulder 24 is shown formed on the inside surface near the distal end of the retraction trunk 20 thereby forming a landing or support for a biased spring 16. A carriage spring ridge or shoulder 25 is shown near the proximal end of the needle carriage 15 wherein the biased spring 16 further thrusts on the needle carriage. The biased spring 16 is restrained from thrusting the needle carriage into the proximal end of the retraction trunk 20 by a stop bar 26. Stop bar 26 is shown disposed in a stop hole 27 formed on the outside surface of the needle carriage 15. The stop bar 26 extends from the stop hole 27, through the retraction trunk hole 28 (see FIG. 3), where it is rotatably fixed to the first end of the retraction lever 30. The retraction lever 30 is further rotatably fixed to the retraction fulcrum 29 near the second end of the retraction lever. The retraction lever 30 is depressed with a finger or a thumb thereby withdrawing the stop bar 26 out of the stop hole 27. The withdrawal of the stop bar 26 releases the needle carriage 15 and allows the biased spring 16 to thrust the needle carriage 15 into the proximal end of the retraction trunk 20.

As the plunger 2 is depressed, the plunger gasket 3 causes an increase in pressure in the medication chamber 5, thereby forcing medication (not shown) or other fluid or gas into the syringe barrel cannula 9, into the carriage cannula 11, into the needle cannula 12, and into a body 19. The carriage seal 10 forms a fluid tight and gas tight seal between the syringe barrel 7 and the needle carriage 15. In a retracted position, a carriage stop 31, formed on the inside surface of the retraction trunk 20, will catch and hold the needle carriage once the needle carriage 15 is released into the retraction trunk.

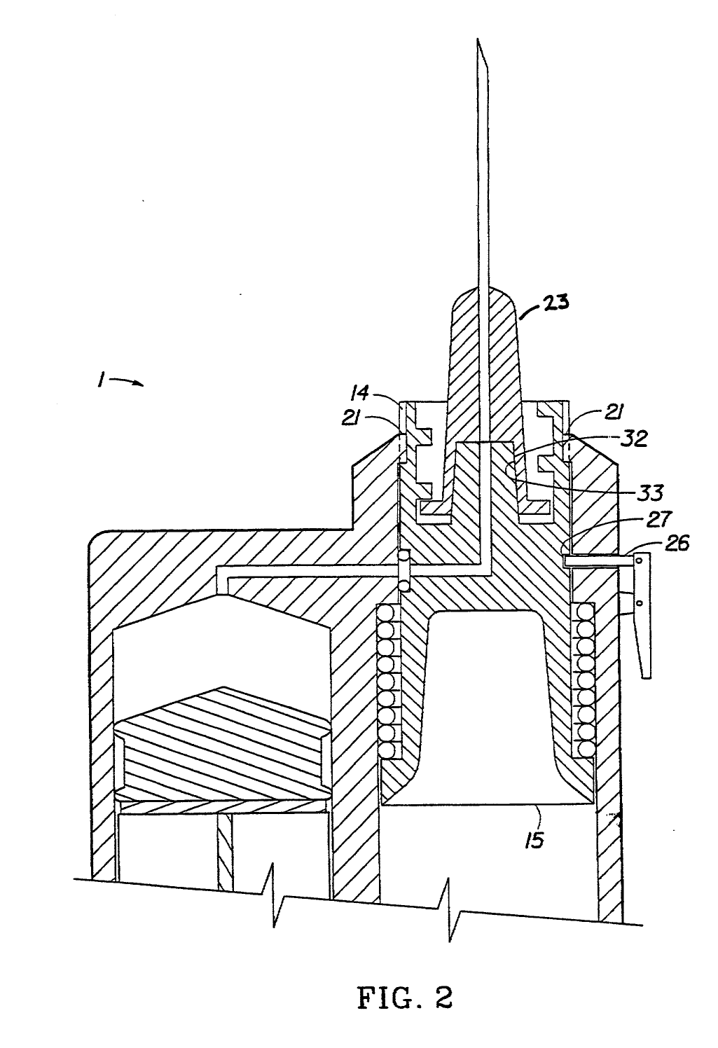

FIG. 2 is an enlarged section elevation of the distal end of the syringe 1 illustrating the locking mechanism between the needle carriage 15 and the retraction trunk 20. The needle carriage 15 has splines 14 which mesh with the splines 21 of the retraction trunk 20. The anti-rotation splines of the needle carriage 15 are shown preventing the needle carriage 15 from rotating while the needle cannula base 23 is being fastened to the needle carriage. A fluid tight seal is shown formed between the carriage cone 32 and the needle cannula foundation 33. This sealing method is commonly referred to as Luer-Lok® (Becton Dickinson, Franklin Lakes, N.J.) in the syringe manufacturing industry. A snap-on fitting or a slip-on fitting could also be used. Furthermore, the stop bar 26 is shown suitably disposed in the stop hole 27 formed in the needle carriage 15.

FIG. 3 is shown a section elevation of the syringe 1 as taken through FIG. 1 illustrating the latching means between the needle carriage 15 and the retraction trunk 20. The first end of the stop bar 26 is shown disposed in the stop hole 27 formed in the outside surface of the needle carriage 15. The center section of the stop bar is shown suitably disposed in the retraction trunk hole 28 formed in the retraction trunk 20. The retraction trunk hole 28 extends from the inside surface to the outside surface of the retraction trunk.

The syringe barrel cannula 9 is shown essentially near the center of the syringe barrel 7. The syringe barrel cannula 9 is shown extending from the syringe barrel 7 into the retraction trunk 20 where it is suitably connected to the carriage cannula 11 formed inside of the needle carriage 15.

FIG. 4 is an enlarged section plan view as taken through FIG. 1 illustrating the seal between the plunger gasket foundation 35 and the syringe barrel 7. The plunger gasket foundation 35 is shown essentially in the center of the syringe barrel 7. The plunger 2 is shown with cross members 36. Although cross members are shown, the plunger could have a cylinder or some other section instead by design choice. Furthermore, the syringe barrel 7 is shown formed or coupled with the retraction trunk 20. The syringe barrel 7 and the retraction trunk 20 could also be bonded by glue or adhesive by design choice. The needle carriage 15 is shown essentially centered within the retraction trunk.

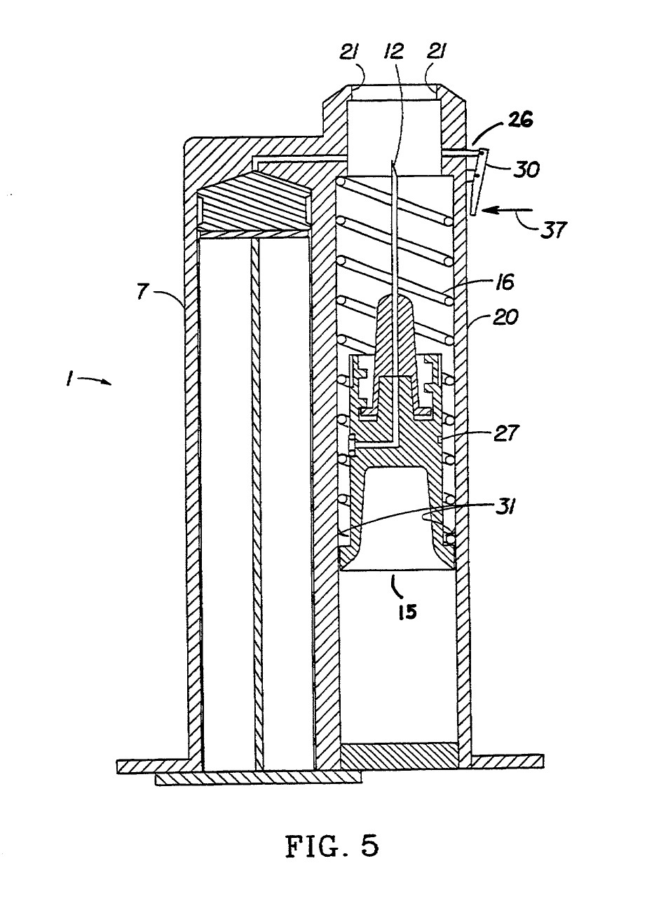

FIG. 5 is a section elevation of the syringe 1 in a retracted position. The retraction lever 30 has been depressed in direction 37, thereby withdrawing stop bar 26 from the stop hole 27 formed in the needle carriage 15. This releases the needle carriage from the stop bar, allowing the biased spring 16 to thrust the needle carriage toward the proximal end of the retraction trunk 20, thereby drawing the needle cannula 12 into the inner confines of the retraction trunk 20. Once inside the retraction trunk, the needle carriage 15 will be caught by the carriage stop 31 and will be retained within the confines of the retraction trunk 20 wherein the now contaminated needle cannula will be unable to prick or otherwise injure a person. Furthermore, the device 1 is shown after medication has been injected into a body, and the plunger has been completely depressed.

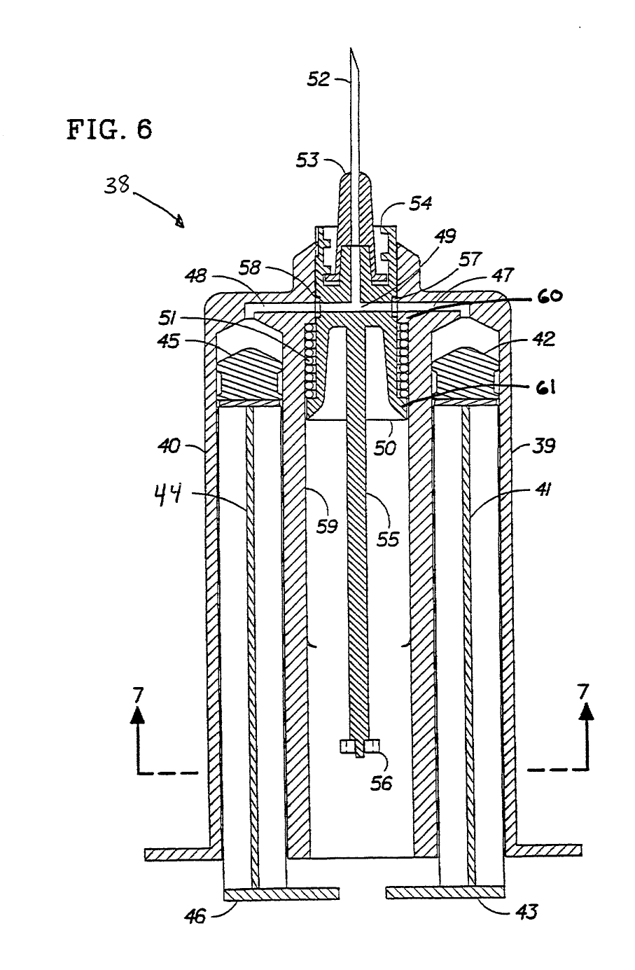

FIG. 6 is a section elevation of a multi-syringe barrel syringe 38 having a first syringe barrel 39 and second syringe barrel 40. Although there are two syringe barrels shown, there could also be more than two syringe barrels by design choice. Although there is only one retraction trunk shown, there could also be more than one retraction trunk by design. The first syringe barrel is shown with a first plunger 41, a first plunger seal 42, and a first thumb flat 43. The second syringe barrel is shown with a second plunger 44, a second plunger seal 45, and a second thumb flat 46. The first and second thumb flats are shown as not being connected to allow one plunger to be depressed independently of the other plunger. A first trunk cannula 47 is shown extending from the first syringe barrel 39 to a carriage manifold 49 formed inside of the carriage 50. A second trunk cannula 48 is shown extending from the second syringe barrel 40 to the carriage manifold 49. A first O-ring 57 is shown forming a gas and fluid tight seal between the first trunk cannula and the carriage manifold. A second O-ring 58 is shown forming a gas and fluid tight seal between the second trunk cannula and the carriage manifold.

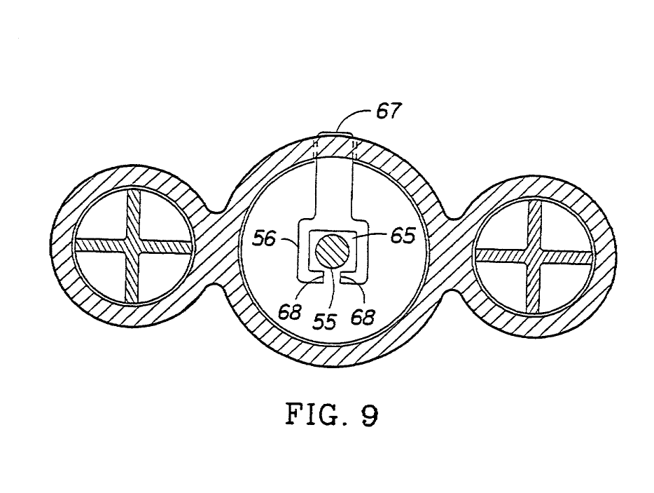

A biased spring 51 is shown disposed between the outside surface of the carriage and the inside surface of the retraction trunk 59. The first end of the biased spring 51 is also disposed on the retraction trunk ridge 60 and the second end of the biased spring is disposed on the carriage ridge 61 formed on the outside surface of the carriage. The biased spring is disposed in a manner to thrust the carriage with the needle cannula into the proximal end of the retraction trunk. In addition, a latch extension 55 is shown extending from the carriage 50. The latch means 56, which will be described in greater details in FIG. 9, secures the carriage in place prior to retraction.

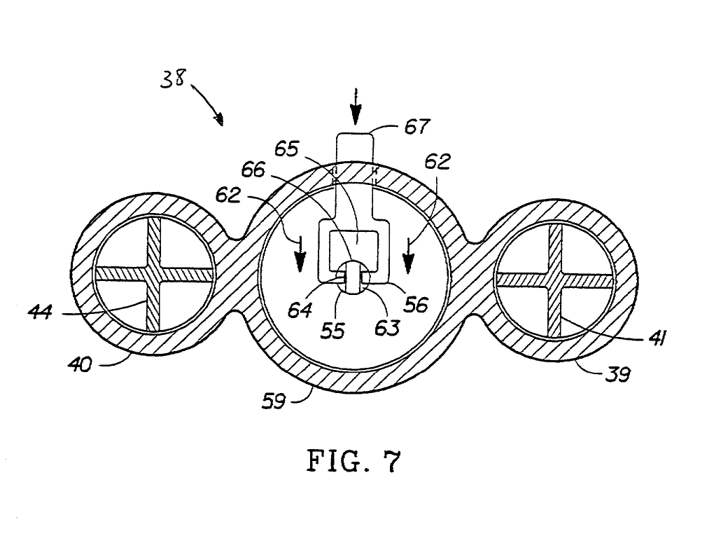

FIG. 7 is a section plan view of the latch release means 56 as taken through FIG. 6. In FIG. 7, the first plunger 41 is shown disposed in the first syringe barrel 39 and the second plunger 44 is disposed in the second syringe barrel 40. Both syringe barrels are shown fixed to the retraction trunk 59.

The latch means 56 is shown restraining the latch extension 55 which in turn restrains the carriage 50 (not shown in this view). A latch extension tab 66 is shown positioned in a latch slot 64 and a latch ridge 63 at the proximal end of the latch extension 55 is shown being retained by the latch means 56. A button 67 is shown at the distal end of the latch means 56 and a latch opening 65 is shown near the inner end of the latch means 56. Depression of the button 67, by a finger or thumb in an inward direction 62, causes the latch opening to be thrust around the latch extension 55 thus releasing the latch extension and the carriage.

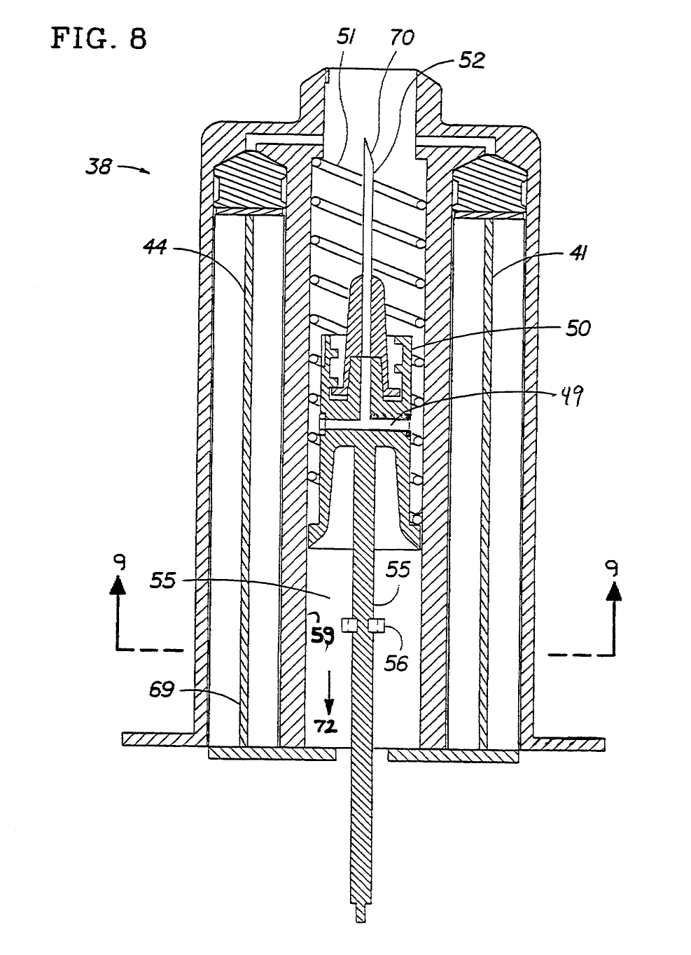

FIG. 8 is a section elevation of the multi-syringe barrel syringe 38 in a retracted position. The figure depicts the latch means 56 as having been depressed, thus releasing the latch extension 55 to move within the retraction trunk 59 in the proximal direction 72. The depression of the latch means 56 allows the biased spring 51 to thrust the carriage 50 with the needle cannula 52 into the retraction trunk 59. Inside the retraction trunk, the point 70 of the needle cannula is sheltered, thereby preventing an accidental needle stick or pricking of others. In addition, the first plunger 41 and the second plunger 44 are shown completely depressed. However, the syringe preferably accommodates either the depression of one or both of the plungers at the user's discretion.

FIG. 9 is a section plan view of the syringe 38 as taken through FIG. 6 with the button 67 in a depressed position. The depression of the button 67 thrusts the latch means 56 off of the latch ridge 63 of the latch extension 55. Furthermore, depression of the button 67 also forces the latch bars 68 off of the latch extension 55 and the latch ridge 63 ( see FIG. 7) and thereby releasing the latch extension.

Although the invention has been described with reference to the preferred embodiments, it will be understood by those skilled in the art, that additions, modifications, substitutions, deletions and other changes not specifically described, may be made in the embodiment herein. It should be understood that the details herein are to be interpreted as illustrations and are not in a limiting sense.

What is claimed is:

1. A retractable safety syringe, comprising:
a syringe barrel forming a fluid chamber;
a retraction trunk fixed to a side of the syringe barrel and having a port in fluid communication with the syringe barrel;
a plunger situated within the syringe barrel, the plunger having a sliding gasket formed along the distal end of the plunger for sealing against the interior of the syringe barrel; and
a biased needle cannula carriage releasably secured within the distal end of the retraction trunk against the port, the biased needle cannula carriage having a distal end including means for selectively receiving a needle cannula and a passage providing fluid communication between the port and the means for selectively receiving a needle cannula.

2. The safety syringe of claim 1, wherein the means for selectively receiving a needle cannula is a connector.

3. The safety syringe of claim 1, wherein the needle cannula carriage is biased by a spring disposed between the outside surface of the carriage and the inside surface of the retraction trunk.

4. The safety syringe of claim 2, wherein the needle cannula carriage is biased in the proximal direction.

5. The safety syringe of claim 2, wherein the needle cannula carriage is biased into the retraction trunk.

6. The safety syringe of claim 1, wherein the needle cannula carriage has smaller cross-sectional dimensions than the interior of the retraction trunk.

7. The safety syringe of claim 5, further comprising a needle cannula base disconnectably connected to the distal end of the carriage base.

8. The syringe of claim 6, wherein the needle cannula base is disconnectably connected by a connector selected from threads, snap-on fittings, slip-on fittings or other means for attachment.

9. The syringe of claim 1, wherein the length of the retraction trunk is greater than the length of the biased needle cannula carriage.

10. The syringe of claim 1, further comprising a stop bar penetrating the wall of the retraction trunk for restricting movement of the needle cannula carriage.

11. The syringe of claim 10, wherein the needle cannula carriage is releasably secured by a stop bar.

12. The syringe of claim 10, wherein the stop bar inserts into the needle carriage.

13. The syringe of claim 10, wherein the stop bar is retracted by a lever.

14. The syringe of claim 1, wherein the retraction trunk is formed with carriage stops.

15. The syringe of claim 14, wherein carriage stops prevent the carriage base from distal movement.

16. The syringe of claim 1, comprising one or more O-rings disposed to form a seal between the syringe barrel and the needle cannula carriage.

17. The syringe of claim 1, wherein the needle carriage is prevented from rotating relative to the retraction trunk.

18. The syringe of claim 1, characterized in that fluids present in the fluid chamber can be delivered to a body through the needle cannula carriage by actuating the plunger.

19. A retractable safety syringe, comprising:
a plurality of syringe barrels;
a retraction trunk fixed to the syringe barrels, wherein the retraction trunk includes passages in fluid communication with the plurality of syringe barrels;
a plunger for each syringe barrel situated within the syringe barrel, the plunger having a sliding gasket formed along the distal end of the plunger for sealing against the interior of the syringe barrel; and
a biased needle carriage releasably secured to the retraction trunk, the biased needle carriage having a means for receiving a needle cannula and a fluid manifold providing fluid communication between the retraction trunk passages and the means for receiving a needle cannula.

20. The syringe of claim 19, wherein the latch means comprises a notched latch extension held in place by a latch.

21. The syringe of claim 19, wherein the latch means is positioned at the proximal end of the retraction trunk.

22. The syringe of claim 19, wherein the latch means comprises a stop bar which penetrates the wall of the retraction trunk and is pivotally connected to a retraction lever.

23. A retractable safety syringe, comprising:
a syringe barrel;
a retraction trunk outside of the syringe barrel that forms an exterior chamber fixed to the barrel;
a plunger situated within the syringe barrel;
a needle carriage held in the distal end by a biasing means and in the proximal end by a latching means and a passage providing fluid communication by means of an O-ring sealed cannula between the needle carriage and the syringe barrel; and
a means for fixing a needle cannula to the distal end of the needle carriage.

24. The syringe of claim 23, wherein the latching means comprises a notched latch extension which is held in place by a latch.

25. A kit of components for assembling safety syringes, comprising:
(a) two or more safety syringe modules having different diameters, each safety syringe module having:
(1) a syringe barrel forming a fluid chamber;
(2) an exterior retraction trunk fixed to a side of the syringe barrel, wherein the distal end of the retraction trunk forms a sealing collar having a port in fluid communication with the syringe barrel;
(3) a plunger extending through a proximal end of the syringe barrel, the plunger having a sliding gasket formed along the distal end of the plunger for sealing against the interior of the syringe barrel; and
(4) a biased needle cannula carriage releasably secured within the distal end of the retraction trunk against the sealing collar, the biased needle cannula carriage having a distal end including means for selectively receiving a needle cannula and a passage through the biased needle cannula carriage providing fluid communication between the sealing collar port and the connector; and
(b) two or more needle cannula modules, each needle cannula module having a needle cannula coupled to a connector, wherein the needle cannula carriages in any of the two or more safety syringe modules are sealably securable to any of the connectors in the two or more needle cannula modules to provide fluid communication between the needle cannula and the needle cannula carriage.

26. The kit of claim 25, wherein the connectors are selected from threads, snap-on fittings, slip-on fittings, or other means for attachment.

27. The kit of claim 25, wherein the needle cannulas of the two or more needle cannula modules are different sizes.
